(19) United States
(12) Patent Application Publication
(10) Pub. No.: US 2009/0157084 A1
(43) Pub. Date: Jun. 18, 2009

(54) COLLAPSIBLE AND EXPANDABLE DEVICE AND METHODS OF USING SAME

(76) Inventors: Arthur Martinus Michael AALSMA, Zutphen (NL); Hubertus Paul Maria Ter Braak, St. Isidorushoeve (NL); Thomas Allard Xander Bonnema, Hengelo (NL)

Correspondence Address: Beusse Wolter Sanks Mora & Maire, 390 N. ORANGE AVENUE, SUITE 2500, ORLANDO, FL, 32801, US

(21) Appl. No.: 12/234,089

(22) Filed: Sep. 19, 2008

Related U.S. Application Data
(63) Continuation-in-part of application No. 11/862,401, filed on Sep. 27, 2007.

(30) Foreign Application Priority Data
Sep. 19, 2007 (GB) 0718200.9

(51) Int. Cl. A61B 17/58; A61M 29/00

(52) U.S. Cl. 606/90; 606/191

(57) ABSTRACT

The present invention provides a device (10) suitable for insertion between vertebral portions and having a first, collapsed, position and a second, extended, position, wherein the device comprises upper and lower supports (18, 20) and side supports (22, 24) pivotally connected thereto and in which one of said side supports includes a reaction surface (34) against which, in operation, an actuation member (36) acts so as to cause opening of said device. The device may be used as an implant or prosthesis or as a tool for compacting diseased bone material. The medical methods of using the device are also disclosed.

The present invention relates to an expandable device having first and second support members and having collapsed and expanded positions together with a mechanism for moving said members apart and relates particularly, but not exclusively, to such a device suitable for use in the repair of defective or damaged bone structures such as, for example, vertebral bodies, disk material between said bodies and the interior portions of other bone structures. The invention also relates to methods of using such a device.

It is known that bone tissue inside, for example, a vertebra deteriorates due to illnesses, such as osteoporosis, trauma and the like, and that any surrounding bone tissue may then be subjected to an ever increasing pressure, which may lead to said surrounding tissue collapsing or the vertebra being compressed, with all the unpleasant consequences thereof. In view of this problem there already exist a number of methods of repairing the deterioration and a number of instruments and prostheses for use in the known methods, some of which are discussed below.

WO 2001/03616, for example, discloses a method of restoring the height of a relatively healthy vertebral body in which upper and lower support plates are coupled to each other by articulated side members hinged at their mid position and operable to assist the upper and lower supports move apart in a substantially parallel relationship. Bone material can be inserted into the space defined by the support so as to further strengthen the supporting structure and restore bone properties.

WO 1998/56301 discloses a method in which the height of a crushed vertebra is restored by inserting an inflatable balloon into the cavity within the vertebra. The balloon is first inserted in a deflated state through a small opening in the vertebral wall and into said vertebral cavity after which it is inflated, as a result of which the vertebra regains its original condition. Then the balloon is deflated and removed, after the space created inside the vertebra is filled with bone cement which, when hardened, restores the mechanical strength of the treated bone. This technique is also described in relation to hip and other joints. One drawback of this method resides in the fact that the inserted material is subjected to a pressure upon deflation of the balloon; as a result of which said material may leak out, so that it will no longer perform its function to its full extent. Furthermore, the quality of the fusion between the inserted material and the surrounding bone tissue may not be fully satisfactory, resulting in a less than optimum long-term strength and quality of the treated vertebra.

WO2003/003951 discloses an instrument for insertion into the vertebral body and includes upper and lower support portions for supporting the vertebra and a mechanism for expanding same such as to restore the vertebral body. The mechanism itself comprises a somewhat complex arrangement of a sliding beam shaped element slidable along a lower surface of the upper support and a pair of parallel side arms which are pivotally arranged relative to the beam element and the lower support such as to allow expansion and contraction of said instrument upon activation of an activation mechanism engageable with one of said parallel side arms. Whilst this arrangement provides a perfectly acceptable mechanism for use in certain applications, the structure thereof is somewhat complex and does not lend itself to use in confined situations.

It is an object of the present invention to provide a device for expanding and compacting the interior of a vertebral body that may be used as an instrument that is removed after bone repair material has been inserted or that may be retained therein as an implant which reduces and possibly eliminates the disadvantages associated with the above-mentioned devices. It is a further object of the present invention to provide a device that may be inserted between vertebral bodies and used as a load bearing device which replaces or partially replaces disk material between said vertebra. It is a still further object of the present invention to provide a device for and method of locking the device in an expanded state and severing connection with any actuation mechanism such as to allow the device to be used as an implant. The present invention also aims to provide a method of inserting an expandable device into a vertebra and expanding it such as to compact diseased bone material therewithin and for allowing the injection of load bearing material such as bone cement around said device such that said device and said material act to at least partially restore the load carrying capacity of the vertebra. Additionally, the present invention provides a method of inserting and expanding an expandable device between vertebra such as to allow the expanded device to carry load between said vertebra

Accordingly, the present invention provides a device for insertion between vertebral portions and having a first collapsed position and a second extended position comprises: a) a first (upper) support member; b) a second (lower) support member; and c) first and second side supports; wherein, said side supports are pivotally connected to said upper and lower members and wherein one or more of said side supports includes a reaction surface against which, in operation, an actuation member reacts to cause opening of said instrument from said collapsed to said extended position.

Preferably, said side supports extend parallel to each other and said first and/or second support member comprises two or more articulated portions.

Advantageously, the combined pivoted length of said first (upper) support and said first side support is substantially equal to the combined pivoted length of said second (lower) support and said second side support and said reaction surface comprises a cam surface against which an actuation member may react upon axial displacement of said reaction member, thereby to cause said side support to pivot about its pivotal connection point and move between a collapsed and an extended position.

In one arrangement said side support includes a second cam surface against which an actuation member may react upon axial displacement of said reaction member, thereby to cause said side support 16 to pivot about its pivotal connection point and move between an extended and a closed position. When provided, said first and/or second cam may comprise two cam surfaces and may be defined by a slot within a side portion of said side support.

Advantageously, said first (upper) support member further includes an extension portion adjacent a pivot point with an associated side support and said extension portion is connectable to an actuation mechanism and said extension portion may be connected to said actuation mechanism by a pivotal connection.

In one arrangement the device includes a lock mechanism for locking said instrument in a position between fully collapsed and fully extended positions which may comprise one or more recesses within one or more of said cam surfaces and into which said reaction member may be lockably located.

Preferably, the device including an actuation member which may comprise an axially translatable member having a surface for engagement with said cam or cams. In a particular arrangement the actuation member includes a carrier portion for carrying said axially translatable member and further includes a locking mechanism for locking said axially translatable member relative to said carrier portion.

Advantageously, the device further includes a separable coupling between said actuation member and said support and still further includes a load sensor for sensing the load exerted on the supports and may also include a load display.

For operator convenience, the device may include a mechanically leveraged trigger mechanism for causing axial translation of an actuation member and such may comprise a hand operable actuation mechanism and a flexible connection between said hand operable mechanism and said supports.

The device may also include a locking mechanism for locking said actuation member relative to said device, such as to prevent further extension or contraction of said device. Whilst various forms of locking mechanism present themselves it has been found that the following are particularly useful and include a locking mechanism which comprises one or more first location features on said actuation member, one or more second location features on said device and an engagement member for engagement between said one or more first location features and said one or more second location features. The first location feature may comprise one or more circumferentially extending indents in said actuation member. Alternatively, said first location feature may comprise one or more circumferentially and axially extending indents in said actuation member. The engagement member itself may be selected from the group comprising: a deformable ring, a spring ring, a Bellville washer, a circlip or an o-ring.

As an alternative to the above, the first location feature may comprise a screw thread on said actuation member and said engagement feature comprises a nut having a threaded portion on an inner surface thereof having a profile corresponding to that of the screw thread on said actuation member for engagement therewith.

The device may further include a locking mechanism for locking said first upper support member relative to a first side member.

In one arrangement said first upper support member and said first side member include threaded portions on outer surfaces thereof and said threaded portions are disposed such as to present a discontinuous but aligned screw thread when said members are in an extended position and the locking mechanism included a ring having a threaded portion on an inner surface thereof engageable with said threaded portions such as to lock said members relative to each other. Said ring may comprise an end portion of a guide sleeve through which, in operation, said device may be inserted.

In a particular arrangement the device includes a head portion and an outer sleeve around said actuation member and said sleeve comprises a plurality of axially extending engagement features on an outer surface thereof and said head portion includes a plurality of corresponding axially extending engagement features on an outer surface thereof for inter-engagement between said engagement features on said sleeve.

Advantageously, said device further includes a splitting mechanism for splitting the actuation member at a point adjacent the head portion. Such a device may include an outer cutter sleeve surrounding the instrument's outer sleeve and having an outlet eccentric to the longitudinal axis X-X and having a cutter edge and wherein the actuation member extends through an off-centre longitudinally extending hole in the outer sleeve and through said outlet such that, in operation, rotation of the outer sleeve causes cutting and separation of the actuation member. Preferably, the outer portions each include handles extending generally perpendicularly to said longitudinal axis X-X.

In a self supporting arrangement said device further includes confronting resting surfaces on said upper support member and said first side support and in which said surfaces are engageable one with the other when said device is in an expanded position and wherein the angle θ between the second lower support member and the second side support is greater than 90 degrees.

Preferably, the device includes a friction lock between said actuation member and said head portion, said friction lock comprising a deformable member expandable in a radial direction upon axial compression thereof. Such a friction lock may comprise a deformable ring around said actuation member and an axially translatable sleeve having a frangible engagement feature thereon which, in operation and upon axial translation thereof engages with said deformable ring to cause radial deformation thereof such as to cause said ring to frictionally engage between the head and said actuation member. Alternatively, said friction lock may comprise a plurality of axially extending resilient fingers on said head, one or more of which includes an engagement portion and in which said actuation member includes a plurality of axially spaced circumferentially extending grooves for receiving said one or more engagement portions upon axial displacement of said actuation member. Preferably, the grooves in said actuation member comprise saw tooth grooves having an incline angled upwardly towards said head portion and wherein said engagement portion on said one or more resilient fingers includes a corresponding inclined portion such that, in operation, said fingers engage with said actuation member such as to ride over said member when said member is pulled such as to expand said device and to prevent said device being retracted.

In one arrangement the device further including a key removably insertable into a split in a split ring such as to maintain said ring in an expanded state when inserted therein thereby to allow said ring to be inserted over said actuation member and removable therefrom such as to cause contraction of said ring and allow engagement thereof within a radially extending groove within said actuation member adjacent said head and with said head portion, thereby to restrict relative movement between said actuation member and said head portion and maintain said device in an expanded state.

The device described above and herein may be used as an implant or prosthesis.

The present invention also provides a method for emplacement of a spacer comprising the steps of: providing a device as claimed in any one of claims 1 to 42; inserting said device in a collapsed state into a structure to be restored; and causing said device to expand within said structure, such as to cause the support members to engage with sound portions thereof, thereby to cause said portions to be moved apart and to a desired distance from each other. The method may include the further step of inserting a bone repair material within a cavity formed by said device and may further include the further step of removing said device from said cavity.

An alternative method for emplacement of an implant comprising the steps of:

providing a device embodiment as described herein; inserting said device in a collapsed state into a structure to be restored; and causing said device to expand within said structure, such as to cause the support members to engage with sound portions thereof.

A still further method for emplacement of an implant between vertebral bodies comprising the steps of: providing a device embodiment as described herein; inserting said device in a collapsed state between said vertebral bodies; and causing said device to expand within said structure, such as to cause the support members to engage with the vertebral bodies. Such a method may also include the step of causing said vertebral bodies to be moved apart through the exertion of a separation force applied through said implant.

Any of the above methods may include the step of disconnecting said actuation member from said device and withdrawing said actuation member, thereby to leave said device within said structure as an implant or prosthesis.

One or more of the methods may also including the step of injecting a load bearing material around an expanded device.

The invention will now be more particularly described by way of example only with reference to the following drawings, in which:

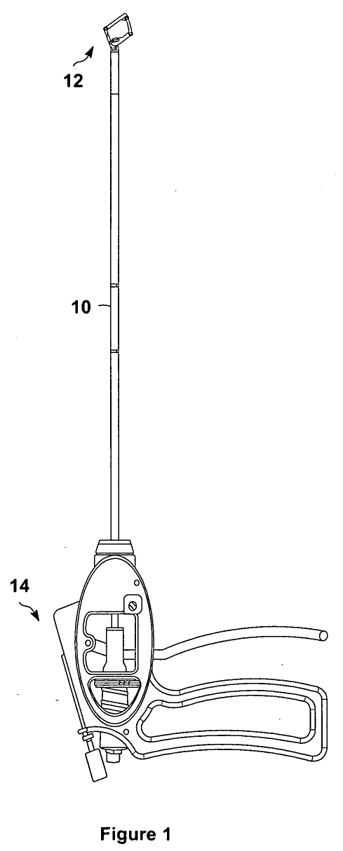

FIG. 1, is a general view of a device according to the present invention when assembled with an actuation mechanism according to another aspect of the present invention so as to form an instrument for use as will be described;

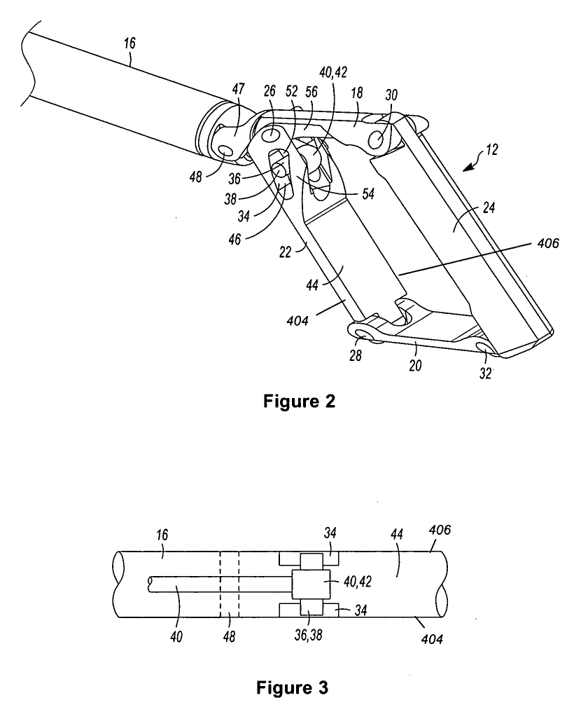

FIG. 2, is an isometric view of the device portion of the present invention;

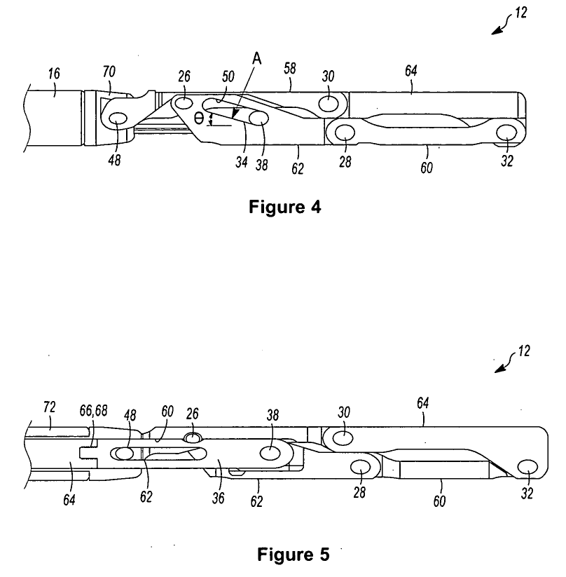

FIG. 3 is a view in the direction of arrow A in FIGS. 2 and 4,

FIGS. 4 to 7, illustrate collapsed, partially expanded and fully expanded positions of the device;

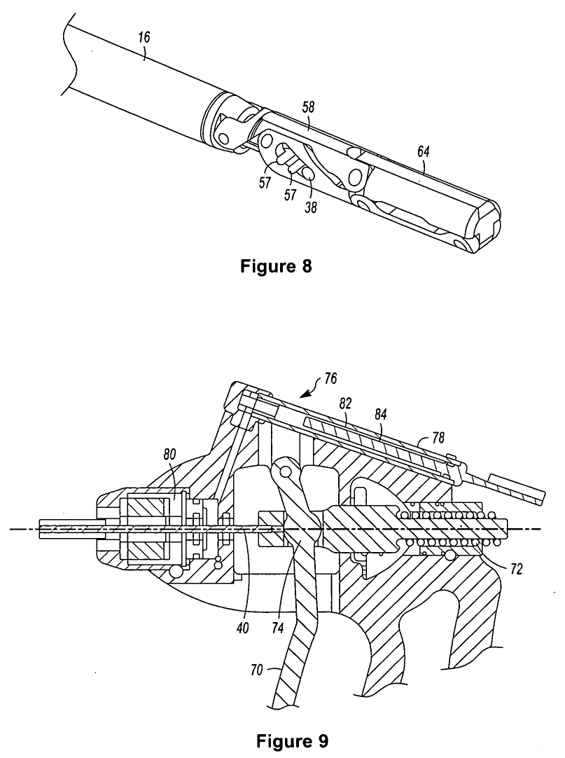

FIG. 8, is an isometric projection of the instrument in its collapsed state;

FIG. 9, is a detailed cross-sectional view of an actuation mechanism suitable for use with the device of FIGS. 1 to 6;

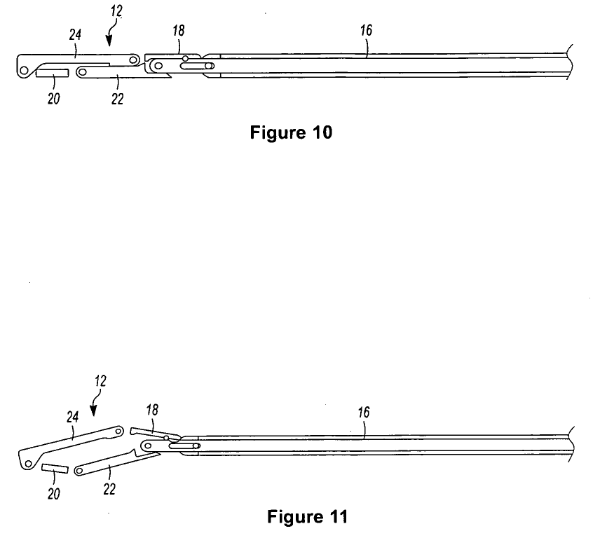

FIGS. 10 to 12 illustrate the tool expanding within a vertebra;

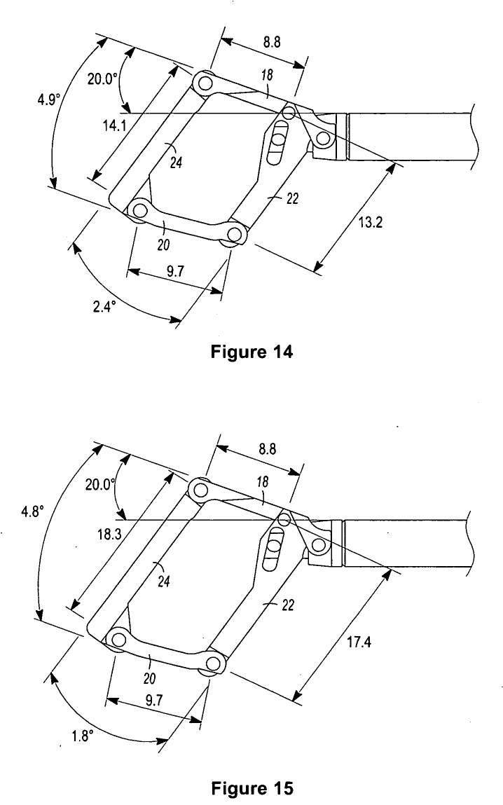

FIGS. 13 to 15 illustrate different dimensional arrangements for the present invention;

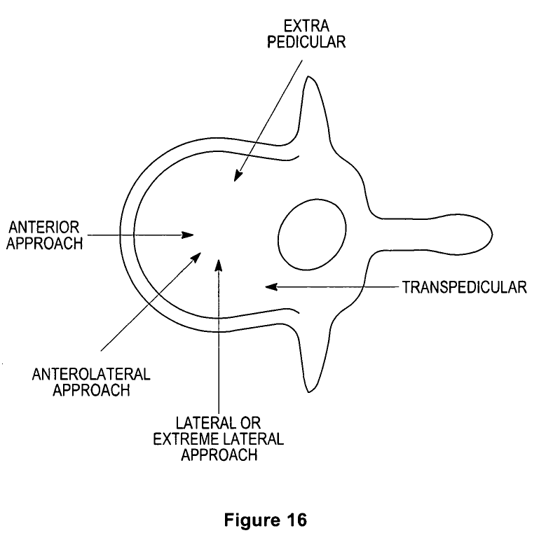

FIG. 16 illustrates a number of possible approach angles that a surgeon may adopt when tackling a collapsed vertebra;

FIGS. 16 to 23 illustrate various forms of locking mechanism for locking the device in a given state;

FIG. 24 illustrates the coupling arrangement between the head of the device and the actuation mechanism;

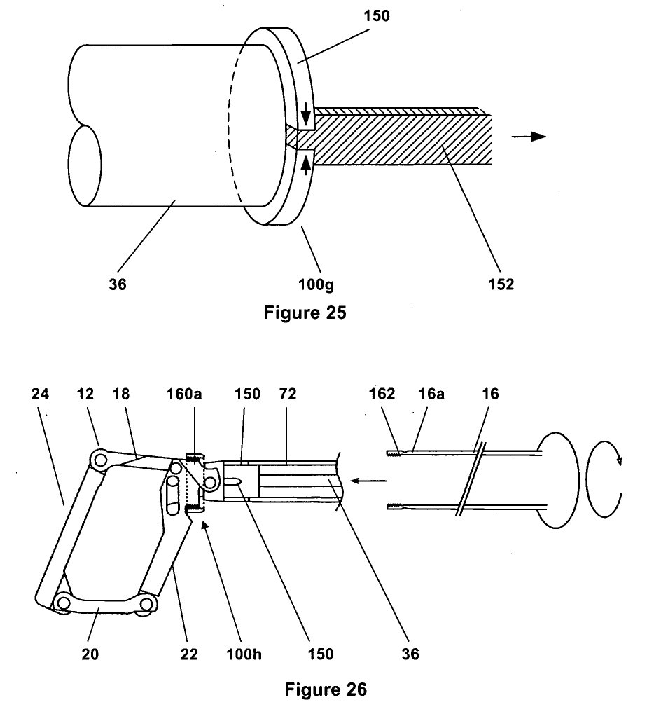

FIG. 25 illustrates a further form of locking mechanism in the form of a spring circlip;

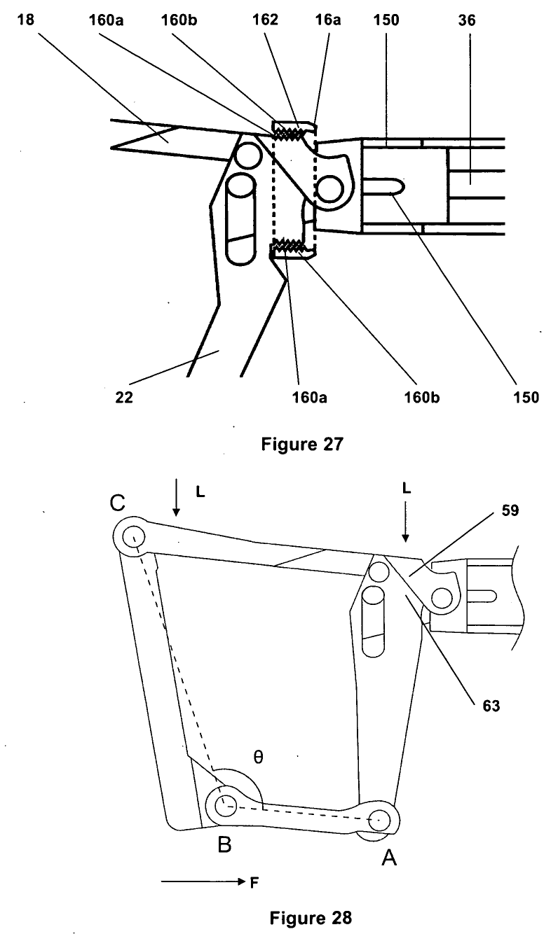

FIGS. 26 and 27 illustrate a still further form of locking mechanism in the form of a threaded nut engageable on two portions of the device itself;

FIG. 28 illustrates a self locking arrangement of the device which employs the applied load L to maintain the device in an expanded state;

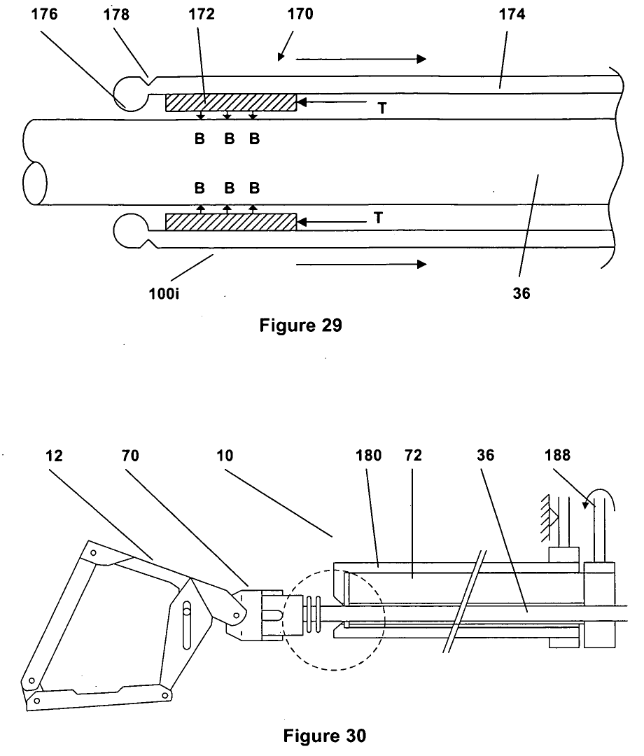

FIG. 29 illustrates a frangible type of locking mechanism;

FIGS. 30 to 32 illustrate a splitting mechanism for splitting the actuation mechanism from the expandable portion of the device;

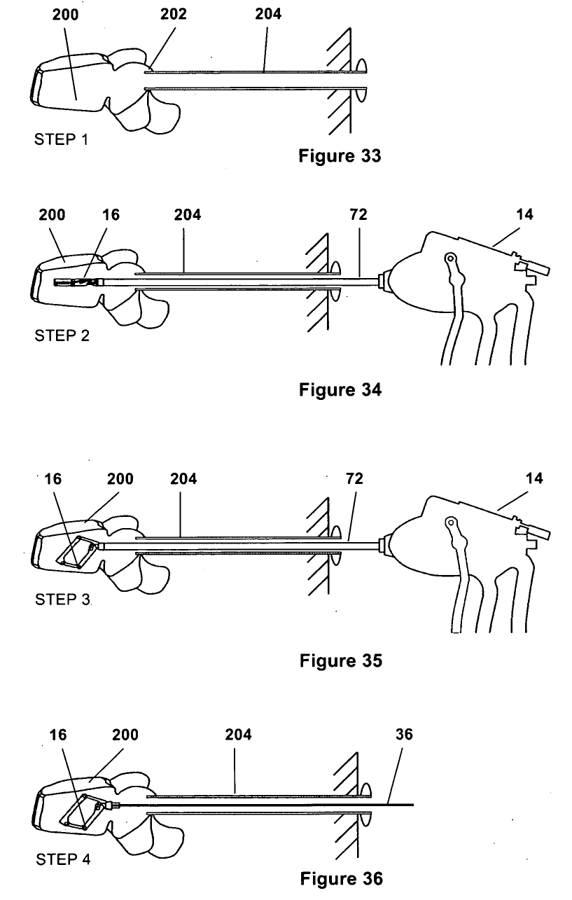

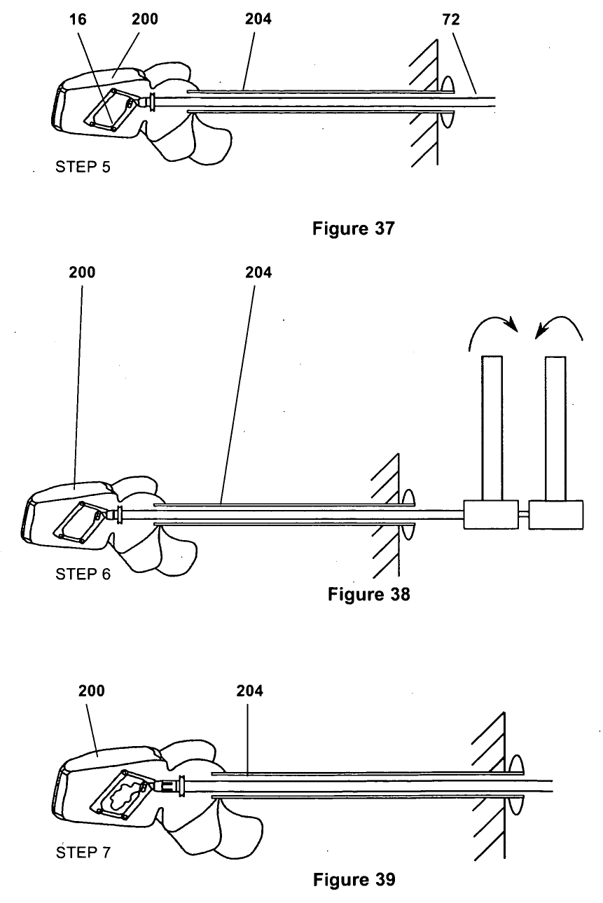

FIGS. 33 to 39 illustrate a method of using the device of FIGS. 1 to 32; and

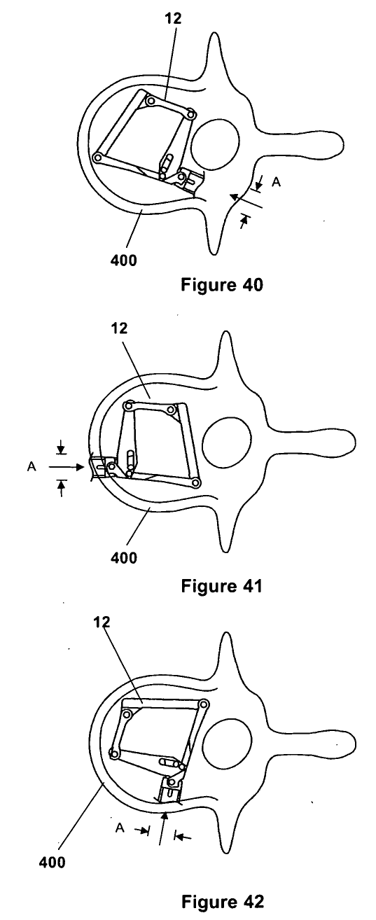

FIGS. 40 to 42 illustrate the device in accordance with an alternative use as an implant between vertebral bodies.

Referring now to the drawings in general but particularly to FIG. 1, an instrument 10 includes a device portion 12 and an actuation portion 14, each of which are shown in more detail in subsequent drawings. The device 12 portion and actuator portion 14 may be separated from each other by an actuation shaft 16 which may be of a rigid or a flexible type, again as detailed later herein.

FIG. 2 illustrates the device in more detail and shows it in a partially expanded state in which upper and lower support members 18, 20 are spaced from each other by load carrying side supports 22, 24 each of which are pivotally connected to said upper and lower support members 18, 20 by means of respective pivots 26, 28, 30, 32, which may be pins or any other suitable pivot arrangement. One or other of the side supports 22, 24 is provided with a reaction surface 34 against which, in operation, an actuation member, a portion of which is shown at 36, may operate. Whilst the actuation member 36 is shown more clearly in later drawings, it will be appreciated that it may include a pin arrangement 38 extending to one or more sides of a central actuation rod 40 which may extend along the actuation shaft 16 to the actuator portion 14 of FIG. 1. The rod itself may comprise a rigid rod or a flexible multi-stranded cable. Either arrangement may be provided with an enlarged head portion 42 through which said pin 38 extends such that it engages with the reaction surface 34. The reaction surface 34 may comprise the inner surface 44 of the appropriate side support 22 or may comprise a cam surface 46 specifically provided thereon, said cam surface 46 being angled upwardly at an angle Θ (FIG. 3) relative to the inner surface 44 so as to assist with deployment as will be discussed later. The first or upper support member 18 includes an extension portion 47 which extends said portion laterally from the upper pivot point 26 towards the actuation shaft 16 and which is provided with a further pivot at 48 which pivotally connects said first (upper) support member to said actuation shaft in a manner that will be appreciated in detail from FIGS. 4 to 8. Further features that may be appreciated from FIG. 2 include the provision of cam surfaces 46 on either side of the actuation rod and the double headed nature of the pin arrangement, which is seen in more detail in FIG. 3. As shown, the device includes optional second cam surfaces 50 provided opposite said first surfaces and against which, in operation, said actuation pins 38 may act so as to contract the assembly. Still further, it will be appreciated that the cam surfaces 46, 50 may be provided as a slot 52 within a raised portion 54 of the side support 18 and that the actuation rod 40 and head portion 42 may pass therebetween in order to correctly position the pin arrangement 38 with respect to said cam surface or surfaces 46. A corresponding portion on the upper support member 18 is wasted or cut away at 56 so as to allow for the assembly to lie one portion within the other, as is shown in the illustration of the collapsed arrangement of FIGS. 4 and 5. Additionally, one or other of the cam surfaces may be provided with a cut-out 57 shaped and dimensioned to correspond with the pin 38 such that said pin can be retained therein by way of an interference fit so as to lock the pin and device in an expanded condition. Finally, the edges of each of the upper and lower supports 18, 20 and side supports 22, 24 may be rounded off as shown by the curved profile of the figures in general, thereby to provide an arrangement in which the cross-sectional profile of the collapsed device is generally circular save for flattened upper and lower support surfaces 58, 60 and side surfaces 62, 64, best seen in FIGS. 4 to 7.

Referring now to FIGS. 4 to 7, which illustrates the device portion extending between a collapsed arrangement and a fully deployed arrangement, it will be appreciated that the profile in the collapsed arrangement is generally slim, with the portions nestling generally one on top of each other with the side supports 22, 24 being positioned in confronting relationship to the upper or lower supports 18, 20. FIG. 3 also aptly illustrates the way in which the wasted portion 58 in the upper support 18 is employed to accommodate the raised side portions 56 which house the cam surfaces 34, 50. The reader will appreciate that pin 38 is positioned at the right hand side of slot 52 and, as long as the pin remains in this position, the device will remain in its collapsed state in which it may be inserted into a vertebral cavity, as will be discussed later herein.

FIG. 5 is a cross-sectional view of the arrangement of FIG. 4 and illustrates in more detail the positioning of the actuation pin 38 within the collapsed side and upper and lower supports. It will be appreciated that in order to accommodate pin 26 it may be necessary to cut a corresponding slot 60 in the actuation member 36 and that said actuation member 36 may be provided with an internal slot 62 for accommodating pivot pin 48 discussed in detail above with reference to FIG. 2. A separable coupling 64 may be provided in the form of corresponding and co-operating threads 66, 68 provided towards the end of actuation member 36 so as to allow a head portion 70 and the device portion 12 to be separated from the actuation mechanism 14, as and when required. In operation, the surgeon need simply twist the rod 40 of the actuation mechanism against the action of the screw thread to cause said rod to disengage from the head portion 70 and allow it to be decoupled, as will be discussed later. The outer sleeve 72 of the actuation shaft is not coupled to the head and is simply held in position by virtue of the screw thread arrangement 66, 68 and its abutment up against the radially extending surface 74 of head portion 70.

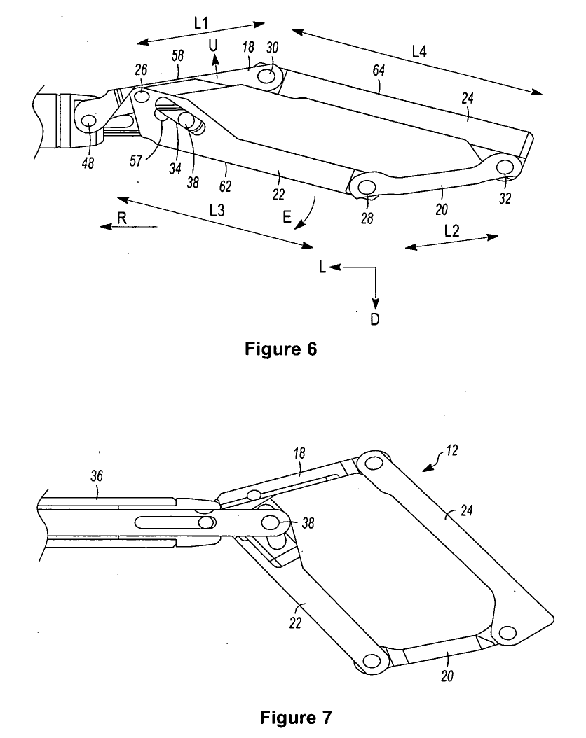

Referring now briefly to FIG. 6, from which it will be appreciated that the device portion 12 may be extended by retracting the actuation rod 36 in the direction of arrow R such as to cause pins 38 to react against cam surfaces 34 and cause the side support 22 to pivot about pin 26 and move in the direction of arrow E to a partially extended position, as shown. The actuation of rod 36 also causes the upper support surface 18 to pivot slightly about pivot pin 48 and move upwardly in the direction of arrow U. Indeed, each of the portions 18, 20, 22, 24 will pivot about their respective pivot pins 26. 28. 30, 32 as actuation rod 36 is moved laterally and this results in the lower support 20 moving generally downwardly in the direction of arrow D and laterally in the direction of arrow L, whilst the upper support 18 simply pivots about pin 48 and moves slightly upwards. It will also be appreciated that most of the motion is experienced by the lower support 20 and the side supports 22, 24 and this heralds an advantage which will be explained later herein.

FIG. 7 illustrates the fully expanded position of the supports, from which it will be seen that the side supports 22, 24 are substantially parallel to each other whilst the upper and lower supports 18, 20 are angled relative to each other by an amount determined by the operable length L1 to L4 between the pivot pins 26, 28, 30, 32. In practice, if the operable length L1 is equal to L2 and operable length L3 equal to L4 the opened device 12 will have parallel upper and lower supports 18, 20 and parallel side supports 22, 24. However, if one alters the various lengths it is possible to alter the angular relationship between the upper and the lower supports 18, 20 such as to provide a slope or incline to the device, which feature may be exploited when attempting to restore a prior or desired angular relationship between affected vertebra. Indeed, this feature in combination with the tilting angle induced by pivoting the upper support about pivot pin 48 endow the present invention with advantages over the art which can assist with the correct positioning of the device and provide improved support for the affected bone portions, both during separation and thereafter. Examples of the various length ratios of the supports 18, 20 and 22, 24 are given in FIGS. 13 to 15, from which it will be appreciated that the critical dimensions are measured between the pivot points or hinge points of each support member relative to the next member. The various lengths can be altered to different finished heights and different finished angles of divergence between the upper and the lower supports 18, 20. In the examples the variation in height is between 13.2 mm of FIGS. 13 and 14 and 17.4 mm of FIG. 15. The angle of divergence is shown to vary between 4.8 degrees and 4.9 degrees, although other angles are possible. This angle is selected to suit the angle of natural alignment of the vertebrae between which the instrument is to be inserted and may change as and when necessary.

FIG. 8 illustrates the device in its collapsed state and illustrates the generally circular profile which allows for the easy introduction thereof down a biopsy needle or the like and also illustrates multiple cut-outs 57 for providing multiple locking positions at different heights.

Turning now to FIGS. 1 and 9 which illustrate one possible actuation mechanism 14, it will be appreciated that the function of this device is to displace the actuation shaft 36 laterally as and when desired in order to cause the pins 38 to react against one or other of cam surfaces 34, 50 so as to open or close the device itself as illustrated in FIGS. 4 to 7. To this end, the actuation mechanism may comprise a simple “push-pull” system such as a loop in the end of the actuation shaft 36 which a surgeon, or assistant, may push, or pull, in order to operate the mechanism 14. Alternatively, a more complex and easily controlled arrangement, such as that shown in FIG. 9, may be provided. The arrangement of FIG. 7 includes a pivotally mounted trigger 70 spring biased by spring 72 and coupled at 74 to the actuation rod 36 such that pulling on the trigger 70 causes the actuation rod 36 to be displaced laterally together with pin 38 so as to cause the opening of the device 12, as described above. Additional features may include a load cell or load detector, shown generally at 76, for determining the amount of operator force being exerted on the device and a visual indicator shown generally at 78. Whilst there exist a number of suitable load cells or detectors and visual indicators that may be used, it has been found that a simple hydraulic chamber 80 and pressure tube 82 having a floating indicator 84 within the tube 82, as shown, may be employed in order to give the operator an indication of the pressure being exerted. The pressure exerted being a direct indication of the separation pressure being exerted on the bone structure concerned. The tube 82 may be marked or calibrated to give a visual indication of the pressure being exerted and may still further be marked with a simple traffic light colouring arrangement with Red indicating excessive pressure, Amber indicating acceptable pressure and Green indicating under loading. Alternatives such as strain gauges and the like may be employed.

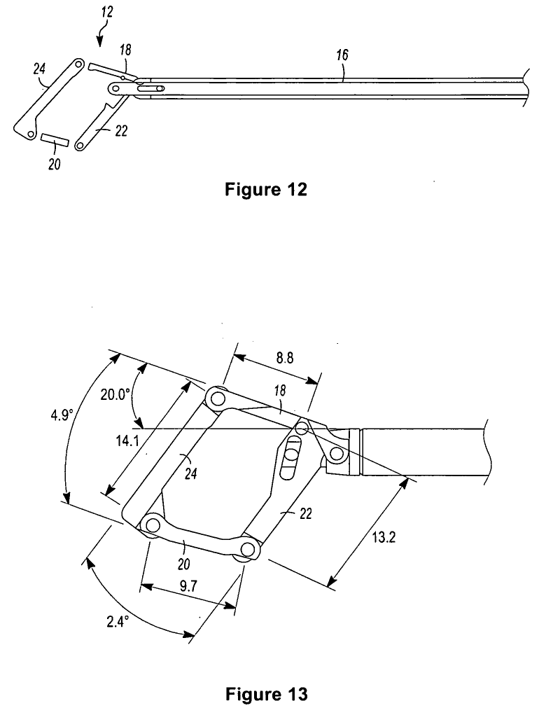

Operation of the above-described arrangement will now be described with reference to FIGS. 10 to 12 and 16, which illustrate the arrangement within a damaged vertebra. In the example shown, the approach is through a small hole previously drilled in the pedicle region and into which a tubular needle (not shown) of internal diameter sufficient to accommodate the present invention is inserted to act as a guide. The device portion is then inserted through the tubular needle such that it emerges from a free end thereof and is positioned between upper and lower damaged or collapsed bone portions 90, 92. A portion of the actuation shaft 16 will remain within the needle which continues to act as both a guide and a support during any subsequent operation. Manipulation of the actuation portion 14 by pulling handle 70 will cause device 12 to expand, as shown progressively in FIGS. 10 to 12. This expansion causes the lower support 20 to move downwardly and backwardly, as described above, which causes the lower bone structure to be progressively pushed downwardly, compressing calcinated material. This movement also pushes the upper and lower portions of the vertebra apart so as open up an inter-vertebral gap, as shown more clearly in FIG. 12. It will be appreciated that whilst the expansion is taking place, the side support 22 will be pivoting about pivot point 26 such that the side support moves downwardly and backwardly in the direction of arrow R in FIG. 6. This action assists with the compaction of any calcinated bone material that might be present in the vicinity and assists with the creation of a stronger and denser surrounding structure. Whilst this compression technique is of particular benefit in the restoration of vertebrae, it may also be used on a number of other surgical where compaction of diseased or wasted bone material is desired. Some of these techniques are described briefly later herein.

FIG. 16 illustrates the various angular approaches that can be employed with the present device, some of which may not be possible with the arrangements of the prior art.

It will be appreciated that the angular relationship or taper between the upper and lower support surfaces can be altered by altering the lengths L1 to L4, as discussed above. Consequently, if one wishes to provide a support having an angular relationship between these two surfaces one simply need alter the lengths L1 to L4 accordingly. Such a feature is of particular benefit when attempting to restore the structure of a vertebra as it may be used to assist with the recreation of the original relationship between the upper and lower portions of the vertebra rather than simply create a parallel association.

Once the device 10 has been fully expanded the supports 22, 24 act to maintain the distance between separated bone portions whilst also acting to take or share any load passed therebetween. Consequently, the device may be decupled from the actuation portion by disengaging screw threads 66. 68, as described above, and withdrawing the actuation shaft 16 such that the device itself becomes a support implant or prosthesis around which the surgeon may insert optional bone repair material. Alternatively, the surgeon may insert such material before withdrawing the device and allowing the inserted material to take any load.

The above device also lends itself to use in the restoration of other bone structures such as, for example, the inter-vertebral gap in which spinal disk material is present. Indeed, the device may be used to separate the vertebrae on either side of an affected disk such as to allow the disk to be removed, repaired, manipulated or replaced before being withdrawn so as to restore the spine to it's pre-damaged state. Still further, the expansion of the device 10 may be employed in the compacting of calcinated bone material within a bone cavity, such as may be present in a femour of a patient suffering from osteoporosis. In such an arrangement, the device 10 may be inserted into the cavity and repeatedly expanded and contracted as it is withdrawn along the cavity, such as to cause compaction of the inner bone material towards the outer portions of the bone itself. Rotation of the device 10 as it is being withdrawn will further assist with the compaction of material. This compaction process may then be followed by the injection of bone repairing material into the created cavity such as to assist with the creation of a stronger bone structure.

It will be appreciated that whilst the above device has been described with reference to an actuation mechanism working against a side support, such a support may form an upper or a lower support depending on the angle of use.

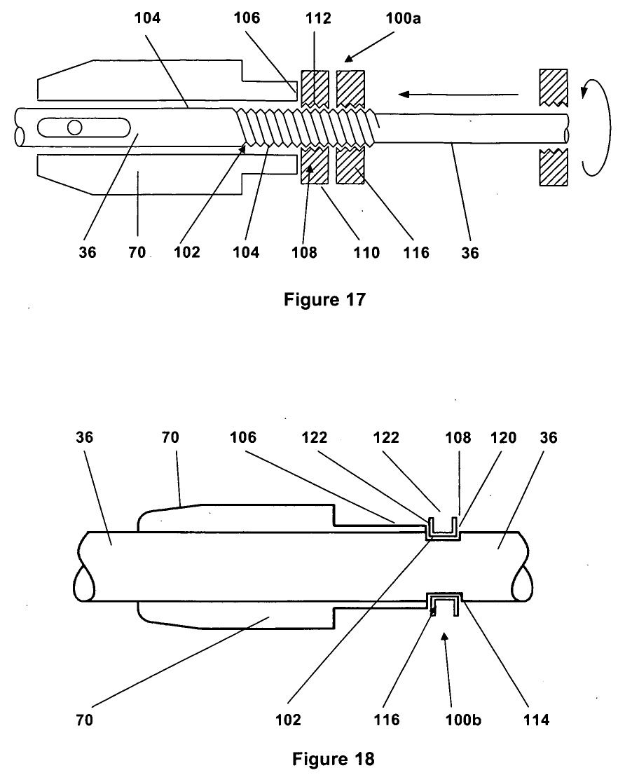

FIG. 17 is a cross-sectional view of a portion of an instrument/implant according to the present invention and provides a first form of locking mechanism 100a for locking the actuation member 36 relative said device portion 12. The locking mechanism includes a first location feature 102 on the actuation member shown in the form of a circumferentially and axially extending screw thread. Also provided is a second location feature 106 on the head 70 of the device itself and an engagement feature 108 in the form of a nut 110 having internal screw threads 112 is engageable with the screw thread 104 and an outer portion 114 engageable with the head portion 70. In operation, the instrument is locked in a desired position by simply sliding nut 110 along the locking mechanism 36 and winding it down the threaded portion 104 such that it engages both the threaded portion and the head portion and prevents relative movement thereof. A second nut 116 may be provided as a locking nut and is placed in its operable position in the same manner as first nut 110.

An alternative form of locking mechanism 100b is shown in FIG. 18 which provides the first location feature 102 in the form of a circumferentially extending groove 118 and the second location feature 106 in the form of a portion of the head 70 again. The engagement feature 108 is provided in the form of a flexible ring 120 having, for example, radially extending portions 122 which, in operation, spring out from a retracted position within the guide tube or sleeve 72 such as to allow the ring 120 to engage with the head portion and prevent axial movement thereof.

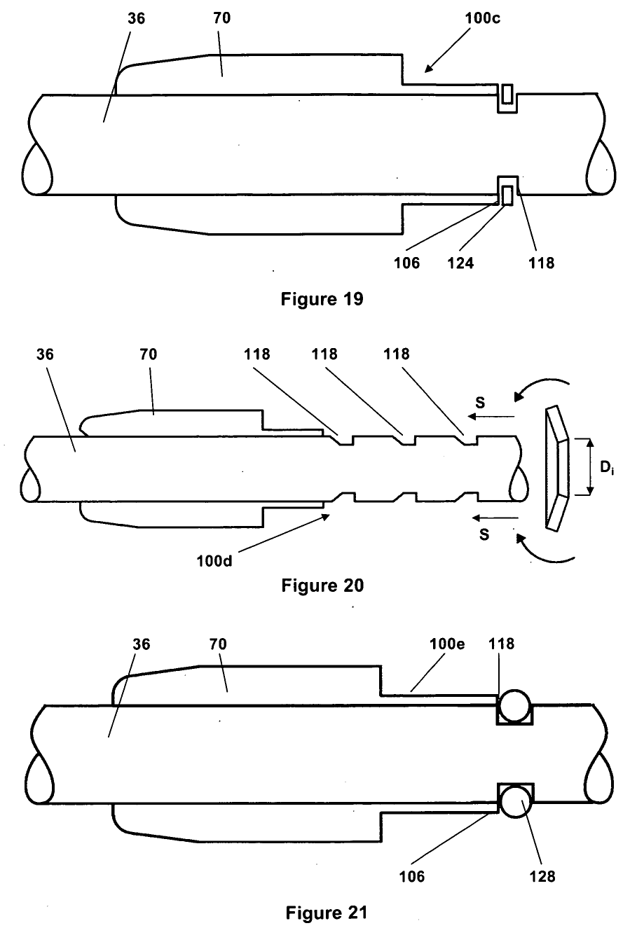

A still further arrangement of locking mechanism 100c is shown in FIG. 19 and in which the flexible ring of FIG. 18 is replaced with a ring or circlip 124 which is retained in a compressed state within the circumferentially extending groove 118 by the external forces applied by the guide tube (not shown) or sleeve 72 and which springs radially outwardly upon removal from said tube or sleeve such as to engage between the groove 118 and a portion of the head 70 of the instrument, as shown.

FIG. 20 illustrates an alternative locking mechanism 100d which is a variation on the arrangement of FIG. 19 and wherein the ring or circlip 124 is replaced with a one-way ring 126 in the form of a biased washer or Bellville washer known in the art. Such a ring forms an arrangement allowing said ring 126 to be slid in the direction of arrow S along the actuation member 36 such that it slides onto one or other of the one or more circumferentially extending grooves 118 provided at discrete positions along the length of the actuation member 36. The Bellville washer is able to slide in the direction of arrow S as it bends inwardly in the direction of arrows B but the tapered nature thereof prevents it sliding in the opposite direction as to do so would mean bending in the opposite direction which is prevented due to the relative stiffness of the ring. Those skilled in the art will appreciate that the inner diameter Dᵢ of the ring 126 is such as to be slightly less than the outer diameter Dₒ of the actuation member 36 and that the rig bends or buckles as it is slid in the direction of arrow S such as to increase the inner diameter thereof. Once in position, the ring 126 locks against the head of the instrument/implant 70 and into groove 118 in the same manner as described above.

FIG. 21 provides a still further locking mechanism 100e and replaces the arrangement of FIG. 19 with an O-ring 128 of expandable but resilient material such as silicon rubber which, in operation is slid along the actuation member such that it contracts back into the circumferential groove 118 and engages between it and the head of the instrument/implant in the manner of FIGS. 18 to 20. Such a ring may be placed in position by providing an outer tube (not shown) which is sized to fit around the actuation member 36 and which is used to force the ring 128 along member 36 by sliding said tube along said member behind said ring 128.

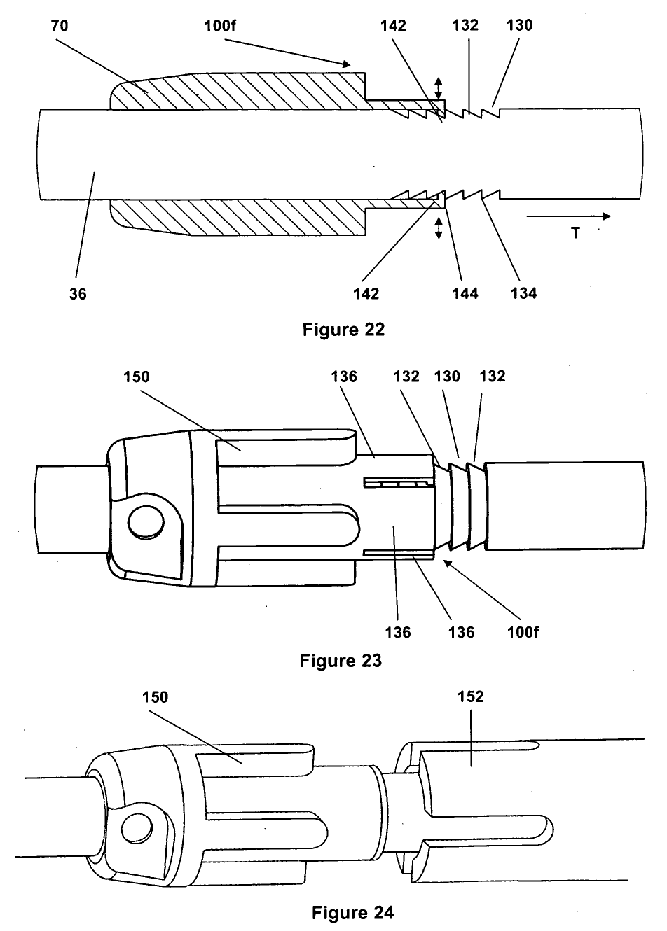

FIGS. 22 and 23 provides a locking arrangement 100f in which the actuation member 36 is provided with a plurality of axially spaced radially extending grooves 130 and the head is provided with corresponding teeth in the manner of a cable-tie. In more detail, the grooves 130 comprise a plurality of saw teeth having an incline 132 angled upwardly (outwardly) towards said head portion 70 and a generally radially extending portion 134 which creates a blocking surface, the function of which will become apparent shortly. A plurality of axially extending resilient fingers 136 also shown in FIG. 23 include a flexible portion 138 and fingers 140 engage with the saw teeth grooves 130 and may be provided with correspondingly profiled sloping surfaces 142 and a bluff surface 144. The reader will appreciate that the slope of the saw teeth provides an effective “one way” engagement feature which, in operation, allows the actuation member 36 to be drawn in the direction of arrow T which allows the fingers to move radially outwardly as they slide over inclined surfaces 132 but prevents the actuation member moving in the opposite direction due to the interference between bluff confronting edges 134 of the tooth and 144 of the finger.

FIG. 24 shows a further detail of the head portion 70 in that it includes a plurality of axially extending and radially displaced torsion fingers 150 on an outer surface of the head portion which, in operation engage with corresponding interposed fingers 152 and provided on the actuation shaft or tube 16 of FIG. 2 above. Such an engagement feature allows the tube 16 which is attached to the operable handle 14 of FIG. 1 to transfer torque or radial motion from the handle 14 to the expandable portion 12 itself whilst also allowing the tube 16 to be disengaged therefrom upon relative axial displacement, as will be appreciated later herein. For purposes of clarity, the gaps between fingers and the teeth of FIG. 23 have been omitted from FIG. 24.

FIG. 25 illustrates a locking arrangement 100g in the form of a circlip locking arrangement in which a circlip 150 is retained in an open position by a key 152 such as to allow it to be slid along actuation member 36 in the manner as described above until it is over a circumferentially extending groove such as described with reference to FIGS. 18 to 20, at which point the key may be withdrawn such that the circlip 150 contracts into said groove and locks itself therein whilst also protruding therefrom such as to engage with the head portion 70, also as described above. Those skilled in the art will appreciate that the ring should be suitably sized such as to crate the desired level of interference when in the locking position.

A different locking mechanism 100h is shown in FIGS. 26 and 27 from which it will be appreciated that a first screw thread 160a is provided on the upper portion 18 and a side portion 22 of the expandable portion of the instrument/implant and a corresponding second screw thread 160b is provided on a ring 162 to be secured therearound. The ring itself is to be threaded onto the first screw thread when the implant is in a fully expanded state such as to engage with the thread thereon and lock the instrument/implant in place. It will be appreciated that such a locking arrangement will only work for one position of the implant/instrument but that, when fully engaged, the screw threads will provide both a secure and a reversible locking arrangement. The ring itself 162 may be an individual ring which can be slid down the actuation member 36 or may form the end of the actuation tube 16 as best seen in FIGS. 1 and 2 and as shown in cross-section in FIG. 23. In such an arrangement the ring plays no significant part until it is desired to lock the arrangement in the fully expanded position. When this is desired, the tube 16 and ring 162 are rotated as one such as to engage the corresponding treads and torque lock the components together. Further application of torque to the tube 16 will cause a frangible portion shown as thinned section 16a to break, thus detaching the ring 162 from the tube 16 and allowing the tube to be removed.

FIG. 28 provides a further locking arrangement 100i in the form of a self locking geometry. In this arrangement the expandable portion of the instrument/implant is modified slightly from that described above in as much as the lengths of the upper and lower and the side supports are such as to allow the angle θ to be greater than 90 degrees and the contact surface 59 and 63 as described briefly above to be fully engaged upon full expansion. In such an arrangement any load L applied to the upper surface will cause side support 22 to attempt to rotate about pivot point C in the direction of arrow F such as to cause contact surfaces 59 and 63 to more fully engage with each other rather than separate. The incline of surfaces 59 and 63 prevents the arrangement from moving further and the instrument/implant is, effectively, locked in its fully expanded position. Clearly, such an arrangement has the advantage of being reversible simply by reversing the direction of force applied to the actuation member 36 such as to collapse the expandable portion and this can be done without the need to first remove the locking member such as would be required in some of the above arrangements.

A still further locking arrangement 100j is shown in FIG. 29 which provides the actuation member 36 with a friction lock in the style of a pop-rivet arrangement. The lock, shown generally at 170, includes a deformable member 172 in the form of a tubular structure surrounding the actuation member 36 and a tubular translation member 174 surrounding the deformable member 172 and engaged therewith at one end by an enlarged head portion 176 having a frangible joint 178 extending around the end thereof and formed as a thinned section. In operation, the deformable member 172 is deformed by pulling the translation member in the direction of arrow T whilst keeping the deformable member 172 stationary relative to the actuation member. This action will cause the deformable member to buckle radially inwardly (arrows B) as it is constrained by the outer actuation member such as to grip the actuation member and lock it in a given position. Upon locking the actuation member will be put under increasing axial load and frangible joint 178 will fail thus allowing the translation member 174 to be withdrawn whilst leaving the enlarged ring head portion 176 in place as a locking ring around the deformable member 172, in the manner of a pop-rivet.

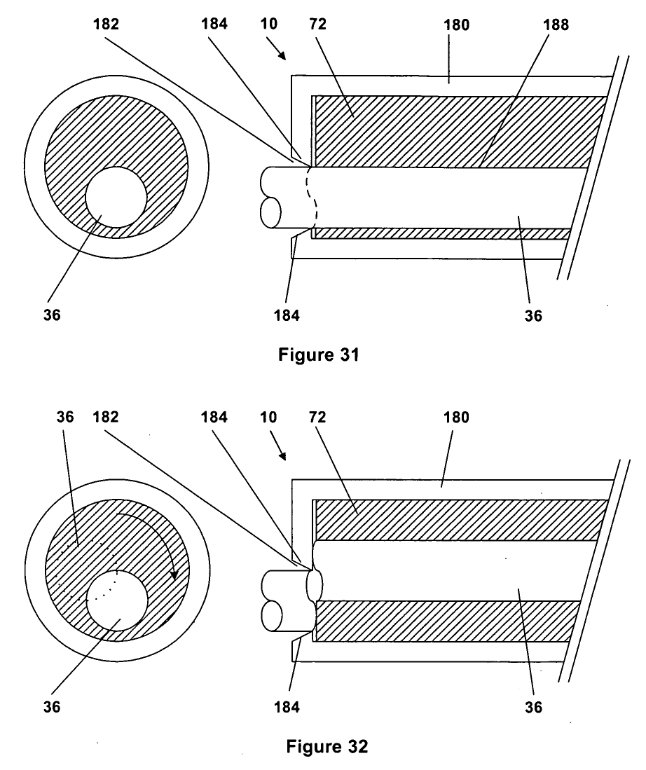

Whilst it will be appreciated to those skilled in the art that there are a number of ways in which the actuation member 36 itself may be severed from the expandable portion 14 including detaching a screw threaded portion (as discussed above) and snapping, we now describe herein a further mechanism in order to assist the reader appreciate the versatility of the present invention and the ease with which it may be converted to an implant, in which the expandable portion is left in the body. Referring to FIGS. 30 to 32, the instrument 10 may be provided with an outer cutter sleeve 180 surrounding the instrument's outer sleeve 72 and which is provided at an open end thereof with an outlet 182 positioned eccentrically to the longitudinal axis X-X. The opening is further provided with a cutter edge formed at 184 and comprising chamfered sharpened edges 186, the operation of which will be described shortly. In this arrangement, the actuation member 36 extends through an off-centre longitudinally extending hole 188 in the outer sleeve 72 and is further provided with a handle or similar mechanism for causing said sleeve 72 to be rotated relative the cutting sleeve 180 as and when desired. When it is desired to sever the expandable portion from the handle portion one simply needs to hold the cutter sleeve 180 still and rotate the outer sleeve 72 of the instrument such as to cause the eccentrically formed cutter hole and cutters to engage with and cut through the actuation member 36, as illustrated diagrammatically in FIGS. 30 and 31.

From the above, the reader will appreciate that the instrument may be converted to an implant as and when desired and when so converted may be left in the patient in order to perform a weight supporting role. A suitable example of such a role is provided in operations in which the interior of a vertebra is to be restored to full or near full weight carrying capacity and in which the instrument as described above may be used to compact diseased bone material within the vertebrae before injecting biocompatible bone material. In such an arrangement the expandable portion 12 could be locked in position by one or more of the above-mentioned locking arrangements and the actuation member severed therefrom by employing one or other of the severing techniques mentioned above before bone material is injected. A full sequence of steps will now be described by way of example and with reference to FIGS. 33 to 39:

In step 1 the vertebral body 200 is prepared for the insertion of the implant 12 by pre-drilling a hole 202 to the correct depth with a suitable drill (not shown) and placing a trocar 204 at the required depth in the pedicle (just outside of the vertebral body). In step 2, the implant 16 is inserted through the trocar 204 and deployed at the desired Anterior-Posterior position (mostly anteriorly near the vertebral wall). Step 3 requires expansion of the expandable portion 16 of the instrument/implant is deployed or expanded, an action which may be repeated several times before being maintained in an expanded position. This step compresses any diseased bone material and forces it up against the more healthy material at the extremities of the vertebra. Step 4 requires that the handle portion 14 and outer sleeve 72 be detached and removed (if necessary), leaving the actuation member or pull wire 36 in position. In step 5 the locking device is introduced or activated and the expandable portion is locked at the desired height. Step 6 requires the expandable portion 12 to be severed from the actuation member 36 in accordance with one or more of the methods as described above such as to form an implant proper. Alternatively, if the implant is locked completely, such as by using the locking nut of FIG. 27, then it may be possible to simply remove the actuation member in its entirety and decouple the actuation rod 36 by unscrewing it rather than severing it. Step 7 comprises the injection of bone cement or other such suitable material for filling the void around the implant and for carrying subsequent load. Injection may take the form of injection down the hole already formed to insert the implant itself or may include the drilling of one or more further holes into the vertebra and terminating just short of the implant itself.

Once the bone cement has set it will act to carry load but also to stabilise the position of the implant which will now also carry load and which is potentially stronger than the bone cement itself and possibly less prone to deterioration over a long period of time.

Those skilled in the art will appreciate that the device mentioned above may be used to compact diseased bone material such as within a vertebra or may be used as an implant within the vertebra where its function may be supplemented with the injection of a load bearing and space filling material such as bone cement as well known to those skilled in the art. In addition to these uses, it is also possible to use the device as an implant between vertebral bodies, as shown diagrammatically in FIGS. 40 to 42 and described in more detail blow.

Referring now more particularly to FIGS. 40 to 41, it will be appreciated that the device described above may be inserted between vertebral bodies 400, 402 so as to act as a load bearing implant. In order to achieve this aim one simply needs to provide an access point or aperture (shown schematically at A) which is large enough to receive the implant 12 in its unexpanded state and then inserting said implant through said aperture A and between said vertebral bodies 400 before expanding said implant 12 such as to cause the sides 404, 406 (see also FIGS. 2 and 3) thereof to contact the mutually confronting faces of adjacent vertebral bodies 400, 402 such as to become load bearing in use. A guide tube or trocar 204 may be used if access is difficult or if desired. FIG. 43 illustrates the juxtaposition of the implant relative the upper and lower vertebral bodies 400, 402.

It will be appreciated that due at least in part to the slender design of the present invention and the fact that it can be expanded in-situ will make introduction and use of such a device easier than many of the prior art arrangements. Further, these features allow the present invention to be used as an implant between vertebral bodies and introduced using approaches that might otherwise be difficult or impossible to implant an implant from. Examples of suitable approaches are shown in FIGS. 40 to 42 which include transpedicular, anterior and extra pedicular approaches. Other approaches such a anteriolateral and lateral or extreme lateral are also possible.

1. A device for insertion between vertebral portions and having a first collapsed position and a second extended position comprises:
a) a first (upper) support member;
b) a second (lower) support member; and
c) first and second side supports;
wherein, said side supports are pivotally connected to said upper and lower members and wherein one or more of said side supports includes a reaction surface against which, in operation, an actuation member reacts to cause opening of said instrument from said collapsed to said extended position.

2. A device as claimed in claim 1, wherein said side supports extend parallel to each other.

3. A device as claimed in claim 1, wherein said first support member comprises two or more articulated portions.

4. A device as claimed in claim 1, wherein said second support member comprises two or more articulated portions.

5. A device as claimed in claim 1, wherein the combined pivoted length of said first (upper) support and said first side support is substantially equal to the combined pivoted length of said second (lower) support and said second side support.

6. A device as claimed in claim 1, wherein said reaction surface comprises a cam surface against which an actuation member may react upon axial displacement of said reaction member, thereby to cause said side support to pivot about its pivotal connection point and move between a collapsed and an extended position.

7. A device as claimed in claim 5 wherein said side support includes a second cam surface against which an actuation member may react upon axial displacement of said reaction member, thereby to cause said side support 16 to pivot about its pivotal connection point and move between an extended and a closed position.

8. A device as claimed in claim 6, wherein said first cam comprises two cam surfaces.

9. A device as claimed in claim 7, wherein said second cam comprises two cam surfaces.

10. A device as claimed in claim 7, wherein said first and second cams are defined by a slot within a side portion of said side support.

11. A device as claimed in claim 1, wherein said first (upper) support member further includes an extension portion adjacent a pivot point with an associated side support and said extension portion is connectable to an actuation mechanism.

12. A device as claimed in claim 9, wherein said extension portion is connected to said actuation mechanism by a pivotal connection.

13. A device as claimed in claim 1 including a lock mechanism for locking said instrument in a position between fully collapsed and fully extended positions.

14. A device as claimed in claim 13, wherein said lock mechanism comprises one or more recesses within one or more of said cam surfaces and into which said reaction member may be lockably located.

15. A device as claimed in claim 1 and including an actuation member.

16. A device as claimed in claim 15 wherein said actuation member comprises an axially translatable member having a surface for engagement with said cam or cams.

17. A device as claimed in claim 16 wherein said actuation member includes a carrier portion for carrying said axially translatable member and further includes a locking mechanism for locking said axially translatable member relative to said carrier portion.

18. A device as claimed in claim 16 and further including a separable coupling between said actuation member and said supports.

19. A device as claimed in claim 16, wherein said actuation member further includes a load sensor for sensing the load exerted on the supports.

20. A device as claimed in claim 19 and further including load display.

21. A device as claimed in claim 1 and including a mechanically leveraged trigger mechanism for causing axial translation of an actuation member.

22. A device as claimed in claim 15 wherein said actuation member includes a hand operable actuation mechanism and a flexible connection between said hand operable mechanism and said supports.

23. A device as claimed in claim 1 wherein said device includes an actuation member for moving said device between said first collapsed position and second extended position and further includes a locking mechanism for locking said actuation member relative to said device such as to prevent further extension or contraction of said device.

24. A device as claimed in claim 1 wherein said locking mechanism comprises one or more first location features on said actuation member, one or more second location features on said device and an engagement member for engagement between said one or more first location features and said one or more second location features.

25. A device as claimed in claim 24 wherein said first location feature comprises one or more circumferentially extending indents in said actuation member.

26. A device as claimed in claim 24 wherein said first location feature comprises one or more circumferentially and axially extending indents in said actuation member.

27. A device as claimed in claim 24 wherein said engagement member is selected from the group comprising: a deformable ring, a spring ring, a Bellville washer, a circlip or an o-ring.

28. A device as claimed in claim 26 wherein said first location feature comprises a screw thread on said actuation member and said engagement feature comprises a nut having a threaded portion on an inner surface thereof having a profile corresponding to that of the screw thread on said actuation member for engagement therewith.

29. A device as claimed in claim 1 and further including a locking mechanism for locking said first upper support member (18) relative to a first side member (22).

30. A device (10) as claimed in claim 29 wherein said first upper support member and said first side member include threaded portions on outer surfaces thereof and said threaded portions are disposed such as to present a discontinuous but aligned screw thread when said members are in an extended position and the locking mechanism included a ring having a threaded portion on an inner surface thereof engageable with said threaded portions such as to lock said members relative to each other.

31. A device as claimed in claim 30 and wherein said ring comprises an end portion of a guide sleeve through which, in operation, said device may be inserted.

32. A device as claimed in claim 1 and wherein said device includes a head portion and an outer sleeve around said actuation member and said sleeve comprises a plurality of axially extending engagement features on an outer surface thereof and said head portion includes a plurality of corresponding axially extending engagement features on an outer surface thereof for inter-engagement between said engagement features on said sleeve.

33. A device as claimed in claim 1 wherein said device further includes a splitting mechanism for splitting the actuation member at a point adjacent the head portion.

34. A device as claimed in claim 33 wherein said splitting mechanism comprises with an outer cutter sleeve surrounding the instrument's outer sleeve and having an outlet eccentric to the longitudinal axis X-X and having a cutter edge and wherein the actuation member extends through an off-centre longitudinally extending hole in the outer sleeve and through said outlet such that, in operation, rotation of the outer sleeve causes cutting and separation of the actuation member

35. A device as claimed in claim 34 wherein said inner and outer portions each include handles extending generally perpendicularly to said longitudinal axis X.

36. A device as claimed in claim 1 and wherein said device further includes confronting resting surfaces on said upper support member and said first side support and in which said surfaces are engageable one with the other when said device is in an expanded position and wherein the angle θ between the second lower support member and the second side support is greater than 90 degrees.

37. A device as claimed in claim 32 including a friction lock between said actuation member and said head portion, said friction lock comprising a deformable member expandable in a radial direction upon axial compression thereof.

38. A device as claimed in claim 37 wherein said friction lock comprises a deformable ring around said actuation member and an axially translatable sleeve) having a frangible engagement feature thereon which, in operation and upon axial translation thereof engages with said deformable ring to cause radial deformation thereof such as to cause said ring to frictionally engage between the head and said actuation member.

39. A device as claimed in claim 37 wherein said friction lock comprises a plurality of axially extending resilient fingers on said head, one or more of which includes an engagement portion and in which said actuation member includes a plurality of axially spaced circumferentially extending grooves for receiving said one or more engagement portions upon axial displacement of said actuation member.

40. A device as claimed in claim 39 wherein the grooves in said actuation member comprise saw tooth grooves having an incline angled upwardly towards said head portion and wherein said engagement portion on said one or more resilient fingers includes a corresponding inclined portion such that, in operation, said fingers engage with said actuation member such as to ride over said member when said member is pulled such as to expand said device and to prevent said device being retracted.

41. A device as claimed in claim 38 and further including a key removably insertable into the split in a split ring such as to maintain said ring in an expanded state when inserted therein thereby to allow said ring to be inserted over said actuation member and removable therefrom such as to cause contraction of said ring and allow engagement thereof within a radially extending groove within said actuation member adjacent said head and with said head portion, thereby to restrict relative movement between said actuation member and said head portion and maintain said device in an expanded state.

42. A device according to claim 1 wherein said device is an implant or prosthesis.

43. A method for emplacement of a spacer comprising the steps of:
providing a device as claimed in claim 1;
inserting said device in a collapsed state into a structure to be restored; and
causing said device to expand within said structure, such as to cause the support members to engage with sound portions thereof, thereby to cause said portions to be moved apart and to a desired distance from each other.

44. A method as claimed in claim 43 including the further step of inserting a bone repair material within a cavity formed by said device.

45. A method as claimed in claim 44 including the further step of removing said device from said cavity.

46. A method for emplacement of an implant comprising the steps of:
providing a device as claimed in claim 1;
inserting said device in a collapsed state into a structure to be restored; and
causing said device to expand within said structure, such as to cause the support members to engage with sound portions thereof.

47. A method for emplacement of an implant between vertebral bodies comprising the steps of:
providing a device as claimed in claim 1;
inserting said device in a collapsed state between said vertebral bodies; and
causing said device to expand within said structure, such as to cause the support members to engage with the vertebral bodies.

48. A method as claimed in claim 47 and including the step of causing said vertebral bodies to be moved apart through the exertion of a separation force applied through said implant.

49. A method as claimed in claim 43 and including the step of disconnecting said actuation member from said device and withdrawing said actuation member, thereby to leave said device within said structure as an implant or prosthesis.

50. A method as claimed in claim 43 including the step of injecting a load bearing material around the device when expanded.

51. A method as claimed in claim 50 wherein said load bearing material comprises bone cement.
